SEQUENCE LISTING (6 sequences)

SEQ ID NO: 1 - DNA, 639 bp, Triticum aestivum  WGA1 (A)
atgaagatgatgagcaccagggccctcgcgctcggcgcggctgccgtcctcgccttcgccgcggcgaccgctcaggcccagaggtgcggcgagcaaggcagcaacatggagtgccccaacaacctctgctgcagccagtacggctactgcgggatgggcggcgactactgcggcaagggctgccagaacggcgcctgctggaccagcaagcgctgcggcagccaggccggcggcgcgacgtgcaccaacaaccagtgctgcagccagtacgggtactgcggcttcggcgccgagtactgcggcgccggctgccagggcggcccctgccgcgccgacatcaagtgcggcagccaggccggcggcaagctgtgcccgaacaacctctgctgcagccagtggggattctgcggcctcggttccgagttctgcggcggcggctgccagagcggtgcttgcagcaccgacaaaccgtgcggcaaggacgccggcggcagagtttgcactaacaactactgttgtagcaagtggggatcctgtggcatcggcccgggctattgcggtgcaggctgccagagtggcggctgcgatggtgtcttcgccgaggccatcaccgccaactccactcttctccaagaatga

SEQ ID NO: 2 - protein, 212 aa, Triticum aestivum WGA1 (A)
MKMMSTRALALGAAAVLAFAAATAQAQRCGEQGSNMECPNNLCCSQYGYCGMGGDYCGKGCQNGACWTSKRCGSQAGGATCTNNQCCSQYGYCGFGAEYCGAGCQGGPCRADIKCGSQAGGKLCPNNLCCSQWGFCGLGSEFCGGGCQSGACSTDKPCGKDAGGRVCTNNYCCSKWGSCGIGPGYCGAGCQSGGCDGVFAEAITANSTLLQE

SEQ ID NO: 3 - DNA, 642 bp, Triticum aestivum WGA2 (D)
atgagaaagatgatgagcaccatggcccttacgctcggcgcggctgtcttcctcgccttcgccgcggcgaccgcgcaggcccagaggtgcggcgagcagggcagcaacatggagtgccccaacaacctctgctgcagccagtacgggtactgcggcatgggcggcgactactgcggcaagggctgccagaacggcgcctgctggaccagcaagcgctgcggcagccaggccggcggggcgacgtgtcccaacaaccactgctgcagccagtacgggcactgcggcttcggagccgagtactgcggcgccggctgccagggcggcccctgccgcgccgacatcaagtgcggcagccagtccggcggcaagctatgcccgaacaacctctgctgcagccagtggggattctgcggcctaggttccgagttctgcggcggtggctgccagagcggtgcttgcagcaccgacaagccgtgcggcaaggacgccggcggcagggtttgcactaacaactactgttgtagcaagtggggatcctgtggcatcggcccgggctattgcggtgcaggctgccagagcggcggctgtgacgctgtctttgccggcgccatcaccgccaactccactcttctcgcagaatga

SEQ ID NO: 4 - protein, 213 aa, Triticum aestivum WGA2 (D)
MRKMMSTMALTLGAAVFLAFAAATAQAQRCGEQGSNMECPNNLCCSQYGYCGMGGDYCGKGCQNGACWTSKRCGSQAGGATCPNNHCCSQYGHCGFGAEYCGAGCQGGPCRADIKCGSQSGGKLCPNNLCCSQWGFCGLGSEFCGGGCQSGACSTDKPCGKDAGGRVCTNNYCCSKWGSCGIGPGYCGAGCQSGGCDAVFAGAITANSTLLAE

SEQ ID NO: 5 - DNA, 561 bp, Triticum aestivum WGA3 (B)
caaaggtgcggcgagcagggcagcggcatggagtgccccaacaacctgtgctgcagccagtacggctactgcgggatgggcggcgattactgcggcaagggctgccagaacggcgcgtgctggaccagcaagcggtgtggcagccaggccggcggcaagacgtgccccaacaaccactgctgcagccagtacgggcactgcggcttcggcgcggagtactgcggcgccggctgccagggcggcccctgccgcgccgacatcaagtgcggcagccaggccggcggcaagctgtgccccaacaacctctgctgcagccagtgggggtactgcggcctcggttccgagttctgcggcgagggctgccagaacggcgcttgcagcaccgacaagccgtgtggcaaggacgccggcggcagggtttgcactaacaactactgctgtagcaagtggggatcctgtggcatcggtcccggctactgcggtgcaggctgccagagcggcggctgcgatggtgtcttcgccgaggccatcgccaccaactccactcttctcgcagaatga

SEQ ID NO: 6 - protein, 186 aa, Triticum aestivum WGA3 (B)
QRCGEQGSGMECPNNLCCSQYGYCGMGGDYCGKGCQNGACWTSKRCGSQAGGKTCPNNHCCSQYGHCGFGAEYCGAGCQGGPCRADIKCGSQAGGKLCPNNLCCSQWGYCGLGSEFCGEGCQNGACSTDKPCGKDAGGRVCTNNYCCSKWGSCGIGPGYCGAGCQSGGCDGVFAEAIATNSTLLAE

(19) United States
(12) Patent Application Publication
(10) Pub. No.: US 2005/0027110 A1
(43) Pub. Date: Feb. 3, 2005

(54) Drug delivery in the nervous system

(76) Inventors: Michael Russell, Davis, CA (US); Yongjin Hou, Davis, CA (US); Carl Cotman, Irvine, CA (US)

Correspondence Address: GRAY CARY WARE & FREIDENRICH LLP, 153 TOWNSEND, SUITE 800, SAN FRANCISCO, CA, 94107, US

(21) Appl. No.: 10/626,351

(22) Filed: Jul. 24, 2003

(51) Int. Cl. A61K 38/18; C07K 14/475; C07K 14/52

(52) U.S. Cl. 530/397; 514/8; 530/351; 530/391.1; 424/178.1

(57) ABSTRACT

The present invention provides compositions useful for transporting agents to, target cells or tissues, e.g., nerve cells via nerve transport. The present invention also provides methods of using the compositions provided by the present invention to deliver therapeutic agents for the treatment of neurologically related disorders.

### FIELD OF THE INVENTION

This invention relates generally to the field of drug delivery, and more specifically to drug delivery for the treatment of neurologically related conditions, e.g. in the central nervous system or other target sites.

### BACKGROUND OF THE INVENTION

Neurological disorders generally include developmental disorders and degenerative disorders of the nervous system. The degenerative disorders usually begin insidiously and run a gradually progressive course over many years. A striking characteristic of the degenerative disorders is that particular anatomic or physiologic systems of neurons may be selectively affected, leaving others entirely intact. This is exemplified in amyotrophic lateral sclerosis, in which the disease process is limited to cerebral and spinal motor neurons, and in some forms of progressive ataxia in which only the Purkinje cells of the cerebellum are affected. In Friedreich's ataxia and some other syndromes, the disease process affects multiple neuronal systems.

In this respect, certain degenerative neuronal diseases resemble others of known cause, particularly intoxications, where similarly circumscribed effects occur. Diphtheria toxin, for example, produces selective breakdown of peripheral nerve myelin, triorthocresyl phosphate affects the cortieospinal, tracts in the spinal cord together with the peripheral nerves, and the neurotoxin 1-methyl-4-phenyl-1,2,3,6-tetrahydropyridine (MPTP) brings about death of dopamine-containing neurons in the substantia nigra.

Typically, the pathologic process in the nervous system is one of slow involution of nerve cell bodies or their axonal extensions, unaccompanied by any intense tissue reaction or cellular response, although the loss of neuron and fibers is often accompanied by hyperplasia of fibrillary astrocytes (gliosis). The cerebrospinal fluid (CSF) shows little if any change-at most a slight elevation of protein, without abnormalities in specific proteins, cell count, or in other constituents.

Neuronal signals are transmitted from cell to cell at specialized sites of contact known as synapses. The usual mechanism of transmission appears surprisingly indirect. The cells are electrically isolated from one another, the presynaptic cell being separated from the postsynaptic cell by a synaptic cleft. A change of electrical potential in the presynaptic cell triggers it to release a chemical known as a neurotransmitter, which is stored in membrane-bounded synaptic vesicles and released by exocytosis. The neurotransmitter then diffuses across the synaptic cleft and provokes an electrical change in the postsynaptic cell. Transmission via such chemical synapses is far more versatile and adaptable than direct electrical coupling via gap junctions, which is also used, but to a much lesser extent. The chemical synapse is a site of intense biochemical activity, involving continual degradation, turnover, and secretion of proteins and other molecules.

The neurons have an efficient intracellular transport system to convey molecules from the cell body (the biosynthetic center of the neuron) to the outermost reaches of the axon and dendrites. In general, nerve transport includes slow and fast transport mechanisms to carry newly synthesized materials from the nerve cell body into the axon and dendrites. Cytoskeletal proteins and many enzymes are carried by slow axonal transport while noncytosolic materials required at the synapse, such as secreted proteins and membrane-bound molecules, move outward from the cell body by a much faster mode of transport. These proteins and lipids pass from their sites of synthesis in the endoplasmic reticulum to the Golgi apparatus, which lies close to the nucleus, often facing the base of the axon. From here, packaged in membrane vesicles, they are carried by fast axonal transport, at speeds of up to 400 mm per day, along tracks formed by microtubules in the axon or the dendrites; mitochondria are conveyed by the same means. Since different populations of proteins are sent out in this way along axons and dendrites, the transported molecules are presumed to be sorted in the cell body into separate and distinctive types of transport vesicles.

The neurons also have an efficient system to allow the nerve terminal to communicate chemically with the cell body, mostly through retrograde transport, e.g., fast retrograde transport of materials back from the ends of the cell processes. The mechanisms of fast transport in the two directions are similar but not identical. The fast retrograde transport has a speed about half that of fast anterograde transport, is driven by a different motor protein, and carries somewhat larger vesicles on average. The structures returning to the cell body consist partly of aging, cytoplasmic organelles, such as mitochondria, and partly of yesicles formed by the extensive endocytosis required for membrane retrieval at the axon terminal after neurotransmitter release. Molecules present in the extracellular medium surrounding the axon terminal are liable to be captured in these endocytic vesicles and thereby carried back from the axon terminal to the cell body.

Nerve transport is the general mechanism by which neurons move large molecules within cell bodies. Nerves have long processes and well developed transport systems to move materials from one end of the cell to another. In some instances molecules can also be moved both within and between cells. Some viruses have evolved the ability to use nerve transport to gain access to the nervous system which is otherwise; well protected against foreign invasion. These neurotrophic viruses can be very specific in the areas which they attack and effect the nervous system, e.g., polio and herpes.

There is a need in the art to provide compositions or methods useful for using nerve transport to deliver agents, especially therapeutic agents for the treatment of neurologically related disorders.

### SUMMARY OF THE INVENTION

The present invention is based on the discovery that certain carbohydrate-binding proteins such as lectins can be used to transport desired agents to neurons or other target cells, e.g., via nerve transport. Accordingly, the present invention provides compositions useful for transporting agents to nerve cells or other target cells and methods for treating, neurologically related conditions.

In one embodiment, the present invention provides a composition useful for nerve transport. The composition includes a transporting entity and a therapeutic agent, wherein the transporting entity is a non-toxic lectin and is operably linked to the therapeutic agent so that the therapeutic agent is capable of being transported to a target.

In another embodiment, the present invention provides a method for treating a neurological condition. The method includes administering to a subject in need of such treatment a therapeutic agent suitable for the treatment of the neurological condition, wherein the therapeutic agent is operably linked to a non-toxic lectin so that the therapeutic agent is capable of being transported to a target associated with the neurological condition.

### DESCRIPTION OF THE PREFERRED EMBODIMENTS

The present invention relates in general to the delivery of agents to desirable targets, e.g., neurons or other cells associated with various neurologically related disorders. It is the discovery of the present invention that certain carbohydrate-binding proteins can take advantage of the nerve transport for moving desired agents to target cells. According to one aspect of the present invention, it provides compositions of a transporting entity operably linked to an agent so that the agent is capable of being transported to desirable targets, e.g., nerve cells or other target cells via nerve transport.

The transporting entity of the present invention can be any carbohydrate-binding protein that is non-toxic to nerve cells. For example, the transporting entity can be a lectin that is non-toxic to neurons: Lectins are proteins that have one or more binding sites for specific carbohydrate sequences and other additional domains capable of interacting with molecules other than carbohydrates in nature. While most lectins have the ability to agglutinate specific types of cells not all lectins are necessarily agglutinins. Lectins are diverse in structure and are characterized by their ability to bind carbohydrates with considerable specificity. In spite of the vast diversity among lectins, however, two aspects of their organization are generally conserved. First, the sugar-binding activity can be ascribed to a limited portion of most lectin molecules, typically a globular carbohydrate-recognition domain (CRD) of less than 200 amino acids. Second, comparison of CRDs reveals that many are related in amino acid sequence.

In general, the non-toxic lectin of the present invention includes all naturally occurring as well as recombinant non-toxic lectins and any functional or structural equivalent thereof, e.g., any lectin modified via amino acid substitution, deletion, insertion, mutation, or chemical modification that does not substantially impair the capability of the lectin to transport desirable agents to target cells. The non-toxic lectin of the present invention also includes lectins modified to reduce its toxicity or increase its compatibility for transporting an agent to target cells, e.g., nerve cells. Usually any lectin that does not substantially damage or interfere with the function of nerve cells can be considered non-toxic to the nerve cells. In a preferred embodiment, the non-toxic lectin of the present invention is capable of being broken down by enzymes in neuronal cells over a period of time, does not interfere with the function of the agent to be transported, or is not mitogenic in nature.

The non-toxic lectin of the present invention can be from various origins, e.g., from plants or animals and can include either a partial or an entire sequence of alectin. In one embodiment, the non-toxic lectin of the present invention is a lectin from Triticum vulgare, i.e. the wheat germ agglutinin (WGA). In another embodiment, the non-toxic lectin of the present invention is APA—Abrus precatorius (jequirity bean), APP—Aegopodium podagaria (ground elder), ABA—Agaricus bisporus (mushroom), ASA—Allium sativum (garlic), Alto A—Allomyrina dichotoma (Japanese beetle), AAA—Anguilla anguilla (fresh water eel), PNA—Arachis hypogaea (peanut), AIA—Artocarpus integrifolia (Brazil jackfruit), AMA—Arum maculatum (lords and ladies), BPA—Bauhinia purpurea (camel's foot tree), BDA—Bryonia dioica (white bryony), CON A—Canavalia ensiformis (jackbean), Succinyl CON A, CCA—Cancer antennarius (California crab), CAA—Caragana arborescens (pea tree), CPA—Cicer arietinum (chick pea), CA—Colchicum autumnale (meadow saffron), CSA—Cytisus scoparius (Scotch broom), DSA—Datura stramonium (jimson weed), DBA—Dolichos biflorus (horse gram), ECA—Erythrina cristagalli (coral tree), EEA—Euonymus europaeus (spindle tree), GNA—Galanthus nivalis (snowdrop bulb); SBA—Glycine Max (soy bean), GS-I—Griffonia simplicifolia, GS-I-A₄, GS-I-B₄, GS-II, HAA—Helix aspersa (garden snail), HPA—Helix pomatia (edible smail), HHA—Hippeastrum hybrid (Amaryllis), HMA—Homarus americanus (lobster), IAA—Iberis amara, IRA—Iris hybrid (Dutch iris), LAA—Laburnum alpinum (Scotch laburnum), LAL—Laburnumn anagyroides (gold chain), LcH—Lens culinaris (lentil), LcH A, LcH B, LFA—Limax flavus (garden slug), LPA—Limulus polyphemus (horseshoe crab), Lotus—Lotus tetragonolobus (asparagus pea), LEA—Lycopersicon esculentum (tomato), MPA—Maclura pomifera (osage orange), MIA—Mangifera indica (mango), NPA—Narcissus pseudonarcissus—(daffodil), OSA—Oryza sativa (rice), PAA—Perseau americana (avocado), LBA—Phaseolus lunatus (lima bean); PHA-L—Phaseolus vulgaris (red kidney bean), PHA-E, PHA-P—, PHA-M—, Phaseolus vulgaris sp. (black bean), PWM, PWA—Phytolacca americana (pokeweed), PEA, PSA—Pisum sativum (garden pea), PMA—Polygonatum mulitiflorum (common Soloman's seal), PTA galactose—Psophocarpus tetragonolobus (winged bean), PTA GalNAc-, RCA-I—Ricinus communis (castor bean), RCA-II—, RPA—Robinia pseudoacacia (black locust), SHA—Salvia horminum, SSA—Salvia sclarea, SNA—Sambucus rigra (elderberry bark), SNA-I—Sambucus nigra (elderberry bark), SNA-II—Sambucus nigra (elderberry bark), STA—Solanum tuberosum (potato) SJA—Sophora japonica (pagoda tree), TKA—Trichosanthes kirilowii (China gourd), TA—Trifolium repens (white clover) WGA—Triticum vulgare (wheat germ), Succinyl WGA, TL—Tulipa, sp. (tulip), UEA-I—Ulex europaeus (gorse, furze), or UEA-II.

In yet another embodiment, the non-toxic lectin of the present invention is a lectin containing the amino acid sequence as shown in SEQ ID NO. 1, SEQ ID NO. 2, or SEQ ID NO. 3. In still another embodiment, the non-toxic lectin of the present invention is a polypeptide containing the minimum amino acid sequence required for a lectin to transport an agent to target cells, e.g., via nerve transport.

According to the present invention, the agent to be transported can be linked to the transporting entity via any suitable means, as known in the art, see for example U.S. Pat. Nos. 4,625,014, 5,057,301 and 5,514,363. For example, the agent to be transported can be covalently conjugated to the transporting entity, either directly or through one or more linkers. In one embodiment, the transporting entity of the present invention is conjugated directly to an agent to be transported. In another embodiment, the transporting entity of the present invention is conjugated to an agent to be transported via a linker, e.g., a transport enhancing linker.

In still another embodiment, the transporting entity of the present invention is conjugated to an agent to be transported via more than one linker, e.g., aminocaproic-horse radish peroxidase (HRP) or a heterobiofunctional cross-linker, e.g., carbonyl-reactive and sulfhydryl-reactive cross-linker. Heterobiofunctional cross reagents usually contain two reactive groups that can be coupled to two different function targets on proteins and other macromolecules in a two or three-step process, which can limit the degree of polymerization often associated with using homobiofunctional cross-linkers. Such multistep protocols can offer a great control of conjugate size and the molar ratio of components.

In general, the transporting enhancing linker of the present invention can be any entity that facilitates the nerve transport process, e.g., via changing the conformation, charge, or any other properties of the resulting molecule so that it is more capable for nerve transport, especially taken up by neuronal cells or across the synaptic barrier. For example, aminocaproic acid is a peptide coupling agent which can be linked with the carbohydrated portion of a lectin and HRP, which leaves the amino groups more exposed on the lectin and results in a negative charge in these molecules. According to the present invention, aminocaproic-HRP is not as effective as HRP with respect to facilitating transportation across the synaptic barrier.

In the event that the agent to be transported is a polypeptide, protein, antibody, or contains amino acids as part of its structure, such agent can be fused either in frame or out of frame with the transporting entity of the present invention, e.g., form a fusion protein. In general, the transporting entity and the agent can be fused directly or via one or more amino acid linkers. Any suitable amino acid linkers can be used to modify the stability, conformation, charge, or other structure features of the resulting fusion protein in order to facilitate its transport to target cells.

According to the present invention, various agents can be transported by the transporting entity of the present invention. In general, the agent to be transported can be any desired entity, e.g., polypeptide, polynucleotide, chemical compound, growth factor, hormone, antibody, cytokine, or the like including entities that cannot pass across the blood-brain barrier by themselves.

Usually, the agent to be transported by the transporting entity of the present invention can be any therapeutic agent useful for treating neuronal cells or other target cells associated with any neurologically related disorder. For example, the agent to be transported by the transporting entity can be a pharmaceutically active agent or a combination thereof that at least as part of its action targets the central nervous system, olfactory, visual system, or any other system associated with neurologically related disorders. The agent to be transported, can also be any imaging agent useful for imaging any neurological pathways or synaptic connections. For example, the agent to be transported can be a diagnostic agent which can be used with an imaging technique such as magnetic resonance imaging (MRI), positron emission tomography (PET), computer-assisted tomography (CAT), X-ray, fluoroscopy and single photon emission computerized tomography.

In one embodiment, the agent to be transported by the transporting entity of the present invention is a neurotrophic factor including, without any limitation, nerve growth factor (NGF), ciliary neurotrophic factor (CNTF), brain-derived neurotrophic factor (DNTF), and glial-derived neurotrophic factor (GDNF). In another embodiment, the agent to be transported by the transporting entity is cardiotrophin-1 (CT1), insulin-like growth factor-1 (IGF1), transforming growth factor-β2 (TGF β2), epidermal growth factor (EGF), fibroblast growth factor (FGF), vascular endothelial growth factor (VEGF), or interferon α.

In yet another embodiment, the agent to be transported by the transporting entity is insulin, glia-derived nexin, gangliosides, phosphatylserine, extracellular matrix remodeling enzymes and their inhibitors, integrins and their ligands, nerve toxins, nerve * transmitters, protein chaperones, or protease inhibitors, e.g., serine protease inhibitors such as 4-(2-aminoethyl)-benzenesulfonyl fluoride (AEBSF). In still another embodiment, the agent to be transported by the transporting entity is not horseradish-peroxidase (HRP).

According to the present invention, the agent of the present invention can be transported to various target cells or tissues. For example, the agent of the present invention can be transported to any nerve cell, e.g., nerve cell in the central nervous system, olfactory, or visual system. The agent of the present invention can also be transported to a neurologically related target cell or tissue, e.g., cells or tissues that interact with or are targets of the nervous system.

In one embodiment, the agent of the present invention is transported to neurons in the brain, especially neurons associated with various neurodegenerative diseases, e.g., Alpers' disease, Alzheimer's Disease, Autosomal Dominant Neurodegenerative Disorder, Batten Disease, Cerebral calcinosis, Cockayne Syndrome, corticobasal ganglionic degeneration, Dementia with Lewy Bodies, Lewy Body Variant, Alzheimers Disease, Motor Neuron Disease, Multiple System Atrophy, Parkinson Plus syndrome, Neuronal intranuclear inclusion disease, Olivopontocerebellar Atrophy, Parkinsonian Syndromes, Pick's disease, Postpoliomyelitis Syndrome, Progressive Supranuclear Palsy, Rett Syndrome, Shy-Drager Syndrome, Tauopathies, Tri-nucleotide repeat diseases, and Tuberous Sclerosis.

In another embodiment, the agent of the present invention is transported to nerve cells associated with neurological disorders, e.g. spinal cord injury, pugilist dementia, pain, neuropathy, neurotrauma, organophosphate poisoning, depression, schizophrenia, anxiety disorders, epilepsy, or bipolar disorder.

In yet another embodiment the agent of the present invention is transported to cells or tissues that interact with or are targets of nerve cells. For example, the agent of the present invention can be transported to muscle cells, glands, or sensory tissues associated with various disease conditions including, but limited to 1) Motor Neuron Diseases, e.g., Anterior Horn Diseases including Poliomyelitis, Amytotrophic Lateral Sclerosis, Spinal Muscular Atrophy (e.g. Werding-Hoffman), 2) Muscle Disorders, e.g., Muscular Dystrophies including Duschenne dystrophy, Becker dystrophy, Limb-Girdle dystrophy, Congenital Dystrophy, Facioscapulohumeral dystrophy, Distal dystrophy, and Oculopharyngeal dystrophy, Necrotizing Myopathies including Polymyositis, and Dermatomyositis, Metabolic Myopathies including Malignant Hyperthermia, Mitochondrial Myopathies, Myotonic Disorders, and Congenital Myopathies, 3) Diseases of the Neuromuscular Junction, e.g., Myasthenia Gravis, and Eaton—Lambert Syndrome, and 4) Diseases of the Peripheral Nerve, e.g., Metabolic Neuropathies including Diabetes Mellitus, Vitamin deficiency, Uremia, and Porphyria, Toxic Neuropathies including alcohol, vincristine, isoniazid, arsenic, lead, hexane, hexachlorophene, acrylamide, and triethyltin, Vasculitic Neuropathies including Polyarteritis nodosa, Churg-Strauss Syndrome, and Rheumatoid arteritis, Inflammatory Neuropathies including Guillain-Barre and Chronic Inflammatory demyelinating neuropathy, Hypertrophic Neuropathies including Charcot-Marie-Tooth Disease, Dejerine-Sottas Neuropathy, and Refsum's Disease, Genetic Neuropathies including the various forms of leukodystrophy, Ataxia-telangiectasia and Giant Axonal Neuropathy, Infectious Neuropathies including Herpes Zoster Neuritis, Herpes Simplex, and Leprosy, Diabetic Neuropathies including Distal symmetrical primarily sensory neuropathy, Autonomic Neuropathy, Proximal asymmetrical painful primary neuropathy, and Cranial mononeuropathy.

In still another embodiment, the agent of the present invention is transported to desired cells or tissues without substantially reaching non-targeted areas, e.g., the agent of the present invention is transported along the nerve pathways or connections via nerve, transport to specific nerve cells or cells interacting therewith.

According to the present invention, the agent of the present invention can be transported directly to nerve cells, e.g., via cell intake such as endocytosis either with or without receptor mediation. The agent of the present invention can also be transported beyond the nerve cells of first contact and continue on to cross synapses in a trans-synaptic manner. In one embodiment, the agent of the present invention can be transported to at least a first, second, or third order neuron distal from the initial cell intake via trans-synaptic transportation. For example, nerve, growth factor can be taken up by the olfactory receptor neurons and transported across at least one, two, or three synapses to reach deep brain structures associated with various neurological diseases.

In general, the nerve transport of the present invention can be any transport via, at least in part a nerve cell or a transport mechanism used by a nerve cell. For example, the nerve transport of the present invention can involve any transport mechanism used by a nerve cell including, without limitation, slow and fast transport mechanisms, anterograde or retrograde fast transport, and synapses. Nerve transport usually provides delivery along existing synaptic connections of the nervous system and allows certain specificity, and predictability of the delivery.

According to another aspect of the present invention, the compositions provided by the present invention can be used to deliver therapeutic agents for the treatment of various conditions associated with neurological disorders. In one embodiment, the compositions of the present invention is provided in pharmaceutical compositions with a suitable carrier. In another embodiment, the compositions of the present invention is provided in a container with a label describing the use of the composition.

In general, the compositions of the present invention can be provided with one or more other non-active ingredients, e.g., ingredients that do not interfere with the function of the active ingredients. For example, the composition of the present invention can include a suitable carrier or be combined with other therapeutic agents.

A suitable carrier can be an aqueous carrier including any safe and effective materials for use in the compositions of the present invention. In one embodiment, an aqueous carrier is used for the compositions of the present invention including, without limitation, thickening materials, humectants, water, buffering agents, surfactants, titanium dioxide, flavoring agents, sweetening agents, coloring agents, and mixtures thereof.

A suitable carrier can also be a pharmaceutically acceptable carrier which is well known to those in the art. Such carriers include, without limitation, large, slowly metabolized macromolecules, e.g., proteins, polysaccharides, polylactic acids, polyglycolic acids, polymeric amino acids, amino acid copolymers, and inactive virus particles.

Pharmaceutically acceptable salts can also be used in the composition, for example, mineral salts such as sodium or stannous fluorides, or sulfates, as well as the salts of organic acids such as acetates, propionates, carbonates, malonates or benzoates. The composition can also contain liquids, e.g., water, saline, glycerol, and ethanol, as well as substances, e.g., wetting agents, emulsifying agents, or pH buffering agents.

In generally, an effective amount of the agents of the present invention to be administered can be determined on a case-by-case basis. Factors to be considered usually include age, body weight, stage of the condition, other disease conditions, duration of the treatment, and the response to the initial treatment.

Typically, the compositions of the present invention are prepared as a topical or an injectable, either as a liquid solution or suspension. However, solid forms suitable for solution in, or suspension in, liquid vehicles prior to injection can also be prepared.

The compositions of the present invention may be administered in any way which is medically acceptable which may depend on the condition or injury being treated. Possible administration routes include injections as well as nasal, ophthalmic, or topical. The compositions may also be directly applied to tissue surfaces. Sustained release, pH dependent release, or other specific chemical or environmental condition mediated release administration is also specifically included in the invention, by such means as depot injections or erodible implants.

In one embodiment, the compositions of the present invention can be administered directly to olfactory mucosa for the treatment of neurological disorders associated with neurons in the brain including deep brain structures.

In another embodiment, the composition of the present invention can be administered via injections to appropriate locations associated with neurological disorders, e.g., injected directly into nerve fibers or into cavities adjacent to or in communication with the target nerve fibers. For example, for treating conditions associated with or accessible by the auditory system, the compositions of the present invention can be injected into the cochlea, i.e., the inner ear and be transported within nerve cells or among nerve cells via trans-synaptic transport, e.g., to the olivocochler system. (See also Vetter et al., Arch. Ital. de Biol., 128:331-353, 1990).

In general, for transport of agents to the spinal cord the compositions of the present invention can be injected directly into a nerve root, nerve fiber or bundle, or into the spino-and acromiodeltoid muscles. (See also Alstermar et al., Exper. Brain Res., 80:83-95, 1990). For transport of agents to the somatosensory system, the compositions of the present invention can be injected into dorsal root ganglion neurons to treat nerve crush injuries. (See also Swett et al., Somatosen. Mot. Res., 12:177-189, 1995). For transport of agents to the visual system, the compositions of the present invention can be injected into the regions of the optic nerve or the retina. (See also Aguayo et al., Ciba Foundation Symposium, Growth Factors as Drugs for Neurological and Sensory Disorders, 196pp. 135-144; discussion 144-148, 1996.)

According to the present invention, the compositions of the present invention can be used to treat various neurologically related disorders, e.g., any disorder that can be appropriately treated by the therapeutic agent delivered by the composition of the present invention. For example, the compositions of the present invention are useful for the treatment of various neurodegenerative disorders including, without limitation, Alpers' disease, Alzheimer's Disease, Autosomal Dominant Neurodegenerative Disorder, Batten Disease, Cerebral calcinosis, Cockayne Syndrome, corticobasal ganglionic degeneration, Dementia with Lewy Bodies, Lewy Body Variant, Alzheimers Disease, Motor Neuron Disease, Multiple System Atrophy, Parkinson Plus syndrome, Neuronal intranuclear inclusion disease, Olivopontocerebellar Atrophy, Parkinsonian Syndromes, Pick's disease, Postpoliomyelitis Syndrome, Progressive Supranuclear Palsy, Rett Syndrome, Shy-Drager Syndrome, Tauopathies, Tri-nucleotide re peat diseases, and Tuberous Sclerosis.

The compositions of the present invention are also useful for the treatment of spinal cord injury, pugilist dementia, pain, neuropathy, neurotrauma, organophosphate poisoning, depression, schizophrenia, anxiety disorders, epilepsy, autism, or bipolar disorder.

### EXAMPLES

The following examples are intended to illustrate but not to limit the invention in any manner, shape, or form, either explicitly or implicitly. While they are typical of those that might be used, other procedures, methodologies, or techniques known to those skilled in the art may alternatively be used.

WGA-NGF conjugates can be delivered to deep brain structures. Further, the experimental data indicate that the growth factor delivered can be kept active and capable of rescuing cells in deep brain structures. Specifically, the data demonstrate that the WGA-NGF conjugates after being intranasally administered in rat can prevent cell death in the medial septum after fimbria fornix lesions.

Conjugation

In the WGA-NGF conjugation procedure, we have used one of carbonyl-Reactive and Sulfhydryl-Reactive cross-linkers, PHPD 3-(2-pyridyldithio)-propionic acid Hydrazide.HCl that contains a carbonyl-reactive hydrazide group on one end and a sulfhydryl-reactive pyridyl disulfide group on the other. WGA was treated with Traut's Reagent (2 iminothiolane.HCl), that reacts with primary amines and introduces a sulfhydryl residue. The NGF carboxyl group is activated by EDC [1-Ethyl-3-(3-Dimethylaminopropyl) carbodiimide Hydrochloride].

The specific procedure is as follows.
1. NGF is resolved in 0.1 M MES, pH 4.8.
2. A 60-fold molar excess of EDC is added.
3. An appropriate volume of 0.1 M PDPH in dry dimethylformamide (DMF) cosolvent is added to yield a 30-fold molar excess. The reaction is allowed to occur for 1 hour.
4. The solution is desalted on a G-25 Sephadex column into 0.1 M sodium phosphate, 5, mM EDTA, pH7.5.
5. 0.1 M Traut's Reagent is added to yield 5-fold molar excess over the WGA for one hour.
6. The column is desalted into 0.1 M sodium phosphate, 5 mM EDTA, pH7.5.
7. Quantitative assays are used to determine the number of pyridyldithio groups and sulfhydryl group introduced onto NGF and WGA, respectively. Approximately 1 mol of thiol reaction group is added per NGF dimer, and two to five thiols are added to each WGA molecule.
8. A 7.5-time molar excess of PDPH derivatized NGF is added to the thiolated WGA and kept overnight at 4° C.
9. The conjugate is dialyzed against 0.1 M sodium phosphate, 5 mM EDTA, pH 7.5, and applied to a WGA affinity Glu-Nac-Gel, and the bound conjugate is eluted with 0.1 M N-acetyl-D-glucosamine.

Wheat germ agglutinin-horse radish peroxidase and other lectins that can be used in this invention are commercially available in various forms and grades. An effectively transported lectin in WGA-HRPd, a form is which the lectin WGA has been bound to the HRP enzyme in a 1:2 ratio and purified and returned to a pH of between 7.2 and 7.4. WGA-HRPd has the advantage of being transported across multiple synapses and degraded within the nervous system. When drug delivery by transport with a conjugated lectin is used as a carrier to bring the said drug into the nervous system, drug side effects are avoided because the conjugated material only travels through the specific nervous pathway to which it has been attached and does not interact with other regions of the brain.

The Transport of Conjugates

The experimental data have suggested that a 50 μl solution of 2% WGA-HRPd can be used as a carrier system for delivery of NGF when applied to the nose of a rat and given at three day intervals. When WGA-HRPd was conjugated to NGF to form WGA-HRPd-NGF and applied in the same fashion, HRP reaction product was found in the identical locations as with the transport of WGA-HRPd alone.

The Transported Agent is Active in Vitro

The biologic activity of the conjugated, WGA-HRPd-NGF in vitro by a standard test was tested by growing PC12 cells (a rat adrenal pheochronocytom cell line) in culture and then applying NGF (now conjugated with WGA-HRPd) to the culture medium and determine if the PC12 cells develop cellular outgrowth. The results of our test with WGA-HRP-NGF revealed biologic activity, e.g., PC12 cells have shown cellular outgrowth upon incubation with WGA-HRP-NGF.

The Transported Agent is Active in Vivo

In addition, we have tested WGA-HRPd-NGF on a group of rats approximately eight months old to determine if the conjugated material would work in vivo by preventing cell death from a surgical lesion. For this test, eight month old rats were divided into two groups. The conjugated WGA-HRPd-NGF was placed in the left nostril of one group and the other group received no treatment as a control. Both groups then received identical surgical lesions that would normally cause secondary cell death. A fimbria-fornix lesion (cutting the axons of cells in the fibmria) was then performed, a procedure that would normally cause cell death by preventing cells from receiving their naturally occurring-trophic factors. This knife cut lesion has been shown to cause the death of cholinergic neurons in septal and diagonal band regions (regions at the center of the brain) after a period of 8-10 days. After the surgery, the rats in the treatment group were given two more applications of the WGA-HRPd-NGF.

Both groups of rats were euthanized after eight days, their brains were perfused and sectioned for histological analysis. The sectioned tissues were placed on glass slides. The slides were then examined for cell survival by staining the tissues with an antibody to choline-acetyltransferase (CAT) antibody. This antibody detects cells protected from cell death while the animals were alive by staining cholinergic neurons. We then performed a “blind” test to determine which specimens exhibited cell survival. The stained slides were placed in a microscope by one person in a manner that did not reveal the animal group identification, and the other person viewed the slides to determine which specimens demonstrated cell survival. The viewing person was readily able to distinguish the control group animals from the experimental group by the presence of healthy cells in the experimental group and the absence of these cells in the control group.

Although the invention has been described with reference to the presently preferred embodiment, it should be understood that various modifications can be made without departing from the spirit of the invention. Accordingly, the invention is limited only by the following claims.

1. A composition useful for nerve transport comprising a transporting entity and a therapeutic agent, wherein the transporting entity is a non-toxic lectin and is operably linked to the therapeutic agent so that the therapeutic agent is capable of being transported to a target.

2. The composition of claim 1, wherein the non-toxic lectin is a lectin from wheat germ agglutinin.

3. The composition of claim 1, wherein the non-toxic lectin is a lectin having the sequence of SEQ ID NO. 1, SEQ ID NO.2, or SEQ ID NO. 3.

4. The composition of claim 1, wherein the agent is a polypeptide, polynucleotide, or compound.

5. The composition of claim 1, wherein the agent is a growth factor, hormone, antibody, or cytokine.

6. The composition of claim 1, wherein the agent does not cross the blood-brain barrier by itself.

7. The composition of claim 1, wherein the agent is a nerve growth factor (NGF).

8. The composition of claim 1, wherein the agent is a Ciliary Neurotrophic Factor (CNTF), glial-derived neurotrophic factor (GDNF), brain derived neurotrophic factor (BDNF), or insulin-like growth factor (IGF1), cardiotrophin-1 (CT1), transforming growth factor-β2 (TGF β2), epidermal growth factor (EGF), fibroblast growth factor (FGF), vascular endothelial growth factor (VEGF) and interferon α.

9. The composition of claim 1, wherein the non-toxic lectin is conjugated with the agent.

10. The composition of claim 1, wherein the non-toxic lectin is fused with the agent.

11. The composition of claim 1, wherein the agent is capable of being transported through nerve transport.

12. The composition of claim 1, wherein the agent is capable of being transported through an olfactory route.

13. The composition of claim 1, wherein the agent is capable of being transported through at least one synapse.

14. The composition of claim 1, wherein the agent is capable of being transported through at least two synapses.

15. The composition of claim 1, wherein the target is a neuron.

16. The composition of claim 1, wherein the target is selected from the group consisting of muscle, gland, and sensory tissue.

17. A method for treating a neurological condition comprising administering to a subject in need of such treatment a therapeutic agent suitable for the treatment of the neurological condition, wherein the therapeutic agent is operably linked to a non-toxic lectin so that the therapeutic agent is capable of being transported to a target associated with the neurological condition.

18. The method of claim 17, wherein the neurological condition is a neurodegenerative disorder.

19. The method of claim 17, wherein the neurological condition is selected from the group consisting of Alpers' disease, Alzheimer's Disease, Autosomal Dominant Neurodegenerative Disorder, Batten Disease, Cerebral calcinosis, Cockayne Syndrome, corticobasal ganglionic degeneration, Dementia with Lewy Bodies, Lewy Body Variant, Alzheimers Disease, Motor Neuron Disease, Multiple System Atrophy, Parkinson Plus syndrome, Neuronal intranuclear inclusion disease, Olivopontocerebellar Atrophy, Parkinsonian Syndromes, Pick's disease, Postpoliomyelitis Syndrome, Progressive Supranuclear Palsy, Rett Syndrome, Shy-Drager Syndrome, Tauopathies, Tri-nucleotide-repeat diseases, Tuberous Sclerosis, spinal cord injury, pugilist dementia, pain, neuropathy, neurotrauma, organophosphate poisoning, depression, schizophrenia, anxiety disorders, epilepsy, and bipolar disorder.

20. The method of claim 17, wherein the non-toxic lectin is a lectin from wheat germ agglutinin.

21. The method of claim 17, wherein the non-toxic lectin is a lectin having the sequence of SEQ ID NO. 1, SEQ ID NO.2, or SEQ ID NO. 3.

22. The method of claim 17, wherein the agent is a polypeptide, polynucleotide, or compound.

23. The method of claim 17, wherein the agent is a growth factor, hormone, antibody, or cytokine.

24. The method of claim 17, wherein the agent does not cross the blood-brain barrier by itself.

25. The method of claim 17, wherein the neurological condition is a neurodegenerative disorder and the agent is a nerve growth factor (NGF).

26. The method of claim 17, wherein the neurological condition is a neurodegenerative disorder and the agent is a Ciliary Neurotrophic Factor (CNTF), glial-derived neurotrophic factor (GDNF), brain derived neurotrophic factor (BDNF), or insulin-like growth factor.

27. The method of claim 17, wherein the non-toxic lectin is conjugated with the agent.

28. The method of claim 17, wherein the non-toxic lectin is fused with the agent.

29. The method of claim 17, wherein the non-toxic lectin is fused in frame with the agent.

30. The method of claim 17, wherein the agent is administered intranasally.

31. The method of claim 17, wherein the agent is capable of being transported through nerve transport.

32. The method of claim 17, wherein the agent is capable of being transported through at least one synapse.

33. The method of claim 17, wherein the agent is capable of being transported through at least two synapses.

34. The pharmaceutical composition comprising the composition of claim 1 and a carrier.

35. A container comprising the composition of claim 1 and a label instructing the use of the composition.
